(19) United States
(12) Patent Application Publication
(10) Pub. No.: US 2026/0083943 A1
(43) Pub. Date: Mar. 26, 2026

(54) CATHETER ASSEMBLY

(71) Applicant: Aperture Medical Technologies Incorporated, Wilmington, DE (US)

(72) Inventors: Shahzada AHMED, Wilmington, DE (US); Akhilesh TRIPATHI, Wilmington, DE (US)

(21) Appl. No.: 18/892,889

(22) Filed: Sep. 23, 2024

(51) Int. Cl. A61M 25/10

(52) U.S. Cl. CPC A61M 25/1018

(57) ABSTRACT

A catheter assembly for use in septoturbinatoplasty procedures. The catheter has a shaft and a primary balloon which comprises a base for positioning against the nasal floor, a roof opposed from the base and a side surface extending therebetween for bearing against a nasal septum and/or an inferior turbinate. The base has a width greater than the roof in a direction transverse to the axis of the shaft. The balloon can be inserted into a patients nasal cavity and inflated to realign the patient's septum and/or treat the turbinate.

### TECHNICAL FIELD

This invention relates generally to septoplasty procedures and inferior turbinate lateralisation procedures (turbinoplasty procedures), a catheter assembly for performing these procedures and a method of doing the same.

### BACKGROUND

The human nasal cavity comprises a nasal septum, a nasal floor and three or sometimes four pairs of turbinates.

A nasal septum is a wall of cartilage and bone that divides the left and right airways of the nasal cavity. A condition known as a deviated nasal septum is when the nasal septum departs from its central position. This condition may be present at birth or caused by trauma to the nose. The nasal turbinates (also known as nasal conchae) are thin sets of plicated walls of bone bilaterally located on either side of the nasal septum arising from the outer nasal walls. In each set of turbinates, a turbinate protrudes inwardly into the nasal passage from either side of the nose, curling in a downwards direction. The turbinates are covered by a layer of vascular tissue (mucosa) which controls the temperature and moisture levels in the nasal passageway. The largest and lowermost of the turbinate pairs are called the inferior turbinates and the smallest and uppermost of the turbinate pairs are called the superior turbinates (or sometimes supreme turbinates if four sets of turbinate pairs are present). The middle turbinates are the pair of turbinates positioned between the inferior and superior turbinates. The vast majority (approximately 90%) of airflow in nasal breathing occurs in the lower one third of the nasal passages.

A deviated septum and/or an enlarged inferior turbinate causes the septum and inferior turbinate to be in closer proximity to one another. This can result in obstruction to the airways, difficulty breathing through the nose, sinus infections, pain and congestion.

A septoplasty procedure is a reconstructive surgery operation to correct a deviated septum. In a typical septoplasty procedure, an incision is made within the nasal cavity to allow access the cartilaginous and bony septum. The mucosal lining that covers the surface of the septum is then lifted away. Some of the bent cartilage or bone is then removed and/or straightened by a surgeon before the mucosal lining is repositioned on the septum and stitched back together. This procedure ordinarily takes around 45 minutes, and requires a high level of skill and expertise to perform. This procedure is also typically combined with inferior turbinate surgery to further increase the volume of the nasal airway. The surgery is commonly performed under general anaesthetic in a theatre environment and can be considered to be invasive and complex.

An enlarged/hypertrophied turbinate may be caused by mucosal inflammation. Typical approaches to reduce the volume of an enlarged/hypertrophied turbinate include resecting a portion of the turbinate using surgery or by utilizing crushing techniques, for example using forceps to lateralise and outfracture the inferior turbinate and increase the volume of the nasal airway.

When the nasal septum becomes deviated, it is common for the mucosa of the inferior turbinate on the opposite side of the nose to become hypertrophied, leading to two blocked nasal passageways, where one nostril is blocked by the deviated septum and the other by the enlarged/hypertrophied turbinate.

Accordingly, it would be beneficial to offer a solution to one or more of the aforementioned problems, by providing a catheter assembly for use in an alternative procedure for the correction of a deviated nasal septum and/or an enlarged/hypertrophied turbinate.

It is an object of the present invention to provide a catheter assembly able to at least partially address one or more of the above issues.

The use of balloon catheters for the treatment of disorders of the nose are known in the patent literature (see US2005/240147A1). However, the reshaping of a septum and/or turbinate in a patient by the act of inflating a balloon between these two anatomical structures is not specified.

All references cited herein are incorporated herein by reference in their entireties.

### SUMMARY OF THE INVENTION

Accordingly, a first aspect of the invention provides a catheter assembly for use in a septoplasty, turbinoplasty and/or septoturbinatoplasty procedure. The catheter assembly may be insertable into a nasal passageway. The catheter assembly may comprise a shaft. The catheter assembly may comprise a primary balloon. The primary balloon, when inflated, may comprise a base for positioning against the nasal floor e.g. of a patient. The primary balloon, when inflated, may comprise a roof opposed from the base. The primary balloon, when inflated, may comprise a side surface extending therebetween for bearing against a nasal septum and/or an inferior turbinate. The primary balloon, when inflated, may have a base with a width greater than the roof in a direction transverse to the axis of the shaft.

The catheter assembly may be usable in an inferior turbinate lateralisation procedure.

Advantageously, the primary balloon can be inserted into a nasal passageway and inflated between the septum and inferior turbinate of a patient. This means the catheter assembly can be used to treat a deviated septum or an enlarged/hypertrophied inferior turbinate without the need for an overly invasive and complex surgical procedure. This procedure may be performed under local anaesthetic in an office rather than under general anaesthetic in a operating theater, as is typical for such procedures. This reduces costs and improves patient recovery times.

In embodiments the primary balloon, when inflated, may have a shape that tapers from the base to the roof.

Advantageously, the primary balloon, when inflated, having a base with a width greater than the width of the roof in a direction transverse to the axis of the shaft and/or the primary balloon having a shape that tapers from the base to the roof means that, in use, the base may engage with the nasal floor whilst a side surface bears against the inferior turbinate. In some embodiments, the base sits below the inferior turbinate, whilst the roof sits above the inferior turbinate, and the tapered side surface bearing against the inferior turbinate extends partially below the inferior turbinate, securing the inflated primary balloon in position. This prevents the primary balloon from moving upwards in the nasal cavity during inflation, towards the skull base and brain, thus greatly improves the safety of the catheter assembly. The shaft may be operably connected to the primary balloon.

Additionally, the present invention advantageously provides a single catheter assembly capable of performing both a septoplasty procedure and a procedure for treating an enlarged/hypertrophied inferior turbinate.

The catheter assembly comprises a shaft which defines a shaft axis e.g. through the shaft. The shaft axis may correspond with the lengthwise axis of the primary balloon e.g. they may be coincident. The shaft axis and the lengthwise axis may correspond with the length of the catheter assembly. In some embodiments, the longest dimension of the primary balloon is in the lengthwise axis. In alternative embodiments, the primary balloon may have a width greater than its length i.e. wherein the dimension perpendicular to the lengthwise axis is greater than the dimension in the lengthwise axis.

Advantageously, this allows the primary balloon to be inserted, positioned and inflated in the desired position within the nasal passageway.

In embodiments, the primary balloon, when inflated, may have a lengthwise axis coincident to the axis of the shaft. The primary balloon may, when inflated, have an irregular quadrilateral shaped cross-section orthogonal to its lengthwise axis. The primary balloon may, when inflated, have a trapezium shaped cross-section orthogonal to its lengthwise axis.

The term “irregular quadrilateral” is intended to mean a quadrilateral shape wherein none of the sides are of equal length, and/or none of the internal angles are of equal measure and/or wherein the quadrilateral shape comprises no lines of symmetry.

As used herein, the term “trapezium” is intended to mean a quadrilateral shape having a base and a pair of sides extending from the base at an angle less than 90° (i.e. the sides taper inwardly toward each other), and a roof connecting the opposite ends of the sides. In some alternative embodiments, one of the sides has a 90° angle relative to the base, and the second side has an angle of less than 90°. Typically, a trapezium comprises a pair of parallel sides and a pair of non-parallel sides. A trapezium may be regular (i.e. comprise a line of symmetry) or irregular (i.e. comprise no lines of symmetry). A trapezium may be isosceles (having two sides which are the same length) or scalene (having no sides which are the same length). In some embodiments, the trapezium may have three sides of the same length i.e. wherein one of the parallel sides is the same length as the two non-parallel sides.

In embodiments, when inflated, the primary balloon may comprise a front surface and a rear surface. The front and rear surface may each have a trapezium shape. The primary balloon may comprise a pair of parallel surfaces. The pair of parallel surfaces may define the roof and the base. The roof and/or base may be rectangular, and optionally, square. The primary balloon may comprise a pair of side surfaces. The side surfaces may comprise opposing, non-parallel quadrilateral surfaces. The roof, base, and pair of side surfaces may all extend in the lengthwise direction. The front and rear surfaces may be identical or non-identical.

The primary balloon having a trapezium shaped cross-section when inflated has a number of advantages. Firstly, in use, the two side surfaces of the inflated primary balloon contact and exert pressure on each of the inferior turbinate and the septum during inflation. The compressive force exerted by the inflating primary balloon may cause a laterally deviated septum to be straightened by plastic deformation and/or an enlarged or hypertrophied turbinate to be reduced by crushing.

Additionally, the trapezium shaped cross-section of the primary balloon allows the base to be positioned atop or against the nasal floor (or hard palate), preventing slippage and misalignment of the primary balloon during inflation e.g. due to stabilisation of the balloon on a surface. This improves the reliability and safety of the catheter assembly

In embodiments, the front and rear surfaces may be parallel to one another. In alternative embodiments, the front and rear surfaces may be non-parallel to one another e.g. they may taper inwardly towards the roof.

In embodiments, the primary balloon may have the shape of a trapezoidal prism or a rectangular frustum when inflated. In some embodiments, the rectangular frustum may comprise a square frustum.

As used herein, the term ‘rectangular’ is intended to mean a quadrilateral having four 90° internal angles. The term ‘trapezoidal prism’ is intended to mean a three dimensional shape comprising two parallel identical trapezium surfaces, with four rectangular surfaces connecting the two trapezium surfaces. The term ‘rectangular frustum’ is intended to mean a three dimensional shape comprising two parallel rectangular surfaces, with four trapezium surfaces connecting the two rectangular surfaces, or alternatively, with one rectangular and three trapezium surfaces connecting the two rectangular surfaces.

In embodiments, the shaft may penetrate, and optionally partially extend into, the rear of the primary balloon. The shaft may extend the length of the primary balloon. The shaft may further comprise a leading portion which extends from the front surface of the primary balloon.

In embodiments, the primary balloon, when inflated, may comprise an aperture extending therethrough. The aperture may extend between and through one surface and another opposing surface of the primary balloon. The aperture may extend through the primary balloon in the lengthwise direction when inflated. The aperture may extend from the front surface to the rear surface of the primary balloon. The primary ballon may thus define a ring-shaped cross-section.

Advantageously, the primary balloon comprising an aperture extending therethrough may thus comprise a first opening on one surface of the primary balloon and a second opening on another opposing surface of the primary balloon. In use, inflation of the primary balloon within the nasal passageway of a patient allows nasal breathing through the aperture during a septoplasty procedure, turbinoplasty procedure, and/or septoturbinatoplasty procedure. Further advantageously, the aperture extending through the primary balloon in the lengthwise direction and/or between and through the front surface and the rear surface of the primary balloon when inflated means that the aperture openings remain unobstructed in use by e.g. nasal floor, the inferior turbinate and/or the septum.

The two side surfaces may be rectangular. Optionally they may be square. In alternative embodiments, the two side surfaces may be trapezium shaped. The side surfaces may be identical to each other.

In a further series of embodiments, the side surfaces may be non-identical. For example, the primary balloon may comprise a first side surface which is larger than a second side surface. Additionally or alternatively, one of the side surfaces may be rectangular and the other side surface may be trapezium shaped. In such embodiments, the primary balloon may have a ‘handedness’. This may be advantageous since the primary balloon may be configurable specifically for use in a left or a right nasal passageway of a patient.

The primary balloon, when inflated, may have a base width defined as the width of the base between the two edges connecting the base to the two side surfaces. The base width may be between 10-20 mm, for example 10, 15 or 20 mm, or may be between 12-18 mm, for example 12, 13, 14, 15, 16, 17 or 18 mm.

The primary balloon, when inflated, may have a roof width defined as the width of the roof between the two edges connecting the roof to the two side surfaces. The roof width may be at least 5 mm, at least 6 mm, 7 mm, 8 mm, 9 mm, 10 mm, 11 mm, 12 mm, 13 mm, 14 mm, or 15 mm. The roof width may be between 5-15 mm, for example 5, 10 or 15 mm, or may be between 7-13 mm, for example 7, 8, 9, 10, 11, 12 or 13 mm.

In embodiments, the primary balloon has a length in the lengthwise axis, wherein the length is between 35-65 mm, for example 35, 40, 45, 50, 55, 60 or 65 mm, or may be between 40-60 mm for example 40, 45, 50, 55 or 60 mm, or may be between 45-55 mm for example 45, 50 or 55 mm. The length may be defined as the distance between the front and rear surfaces e.g. at its longest point. The length may be defined as the distance along the base between the two edges connecting the front surface and rear surface.

In embodiments, the primary balloon may have a height defined as the distance between the roof and the base. The height may be between 15-35 mm, for example 15, 20, 25, 30 or 35 mm, or may be between 20-30 mm, for example 20, 25 or 30 mm. The height may be measured orthogonally to the base. The height may be measured from the centre of the base preferably, the height is measured from the centre of the base to the centre of the roof. In embodiments where the roof and base are not parallel, the height of the primary balloon may be the average height of the balloon between the roof and base. It will be understood that the dimensions above refer to the primary balloon when inflated.

Advantageously, the primary balloon having a roof width, means that the primary balloon shape, when inflated, can be designed such that contact between the septum and/or the inferior turbinate with one or more side surfaces can be optimised for an septoplasty procedure, turbinoplasty procedure and/or septoturbinatoplasty procedure whilst keeping the height of the primary balloon to a minimum. This avoids potential force being applied to non-targeted regions the nasal cavity e.g. the roof of the nasal cavity and/or middle and/or superior turbinates.

Upon inflation, if the primary balloon failed to adopt the secured position, or if the primary balloon was to deviate from this secured position upon inflation or otherwise, the risk of harming the patient is much lower. Additionally, the inflated primary balloon having a roof width, as opposed to two side surfaces meeting at an apex, means that if the primary balloon failed to adopt the secured position or was to move upwards during a procedure, the roof area potentially coming into contact with anatomical structures located proximal to the upper part of the nasal cavity would be blunt rather than pointed. Thus, such a balloon is better able to distribute the forces and reduces the potential degree of harm to a patient. The primary balloon when inflated having a roof width improves the safety of the catheter assembly.

As used herein, the term ‘secured position’ is intended to mean the position where the primary balloon is inflated, or partially inflated, with the base of the primary balloon positioned above or against the nasal floor, and wherein a tapered side surface bears against and partially extends below the inferior turbinate or the septum, securing the primary balloon in position.

In embodiments, the front and rear surfaces may be substantially flat. The base and roof may be substantially flat. The two side surfaces may be substantially flat.

In embodiments, the primary balloon may comprise a contrast agent located at one or more edges and/or vertices of the primary balloon. Advantageously, locating the contrast agent at one or more edges and/or vertices of the primary balloon improves the precision of visualisation during surgery.

In embodiments, the primary balloon may comprise a single, unitary dilator. Advantageously, the primary balloon comprising a single dilator is simple to manufacture, reducing the cost of manufacturing the catheter assembly.

In alternative embodiments, the primary balloon may comprise a plurality of dilators. For example, the primary ballon may comprise 2, 3, 4, 5, 6, 7, 8, 9, 10 or more dilators. The plurality of dilators may each be individually inflatable, or alternatively, they may be fluidically connected such that they are simultaneously inflatable. The plurality of dilators may be connected together. The plurality of dilators may each be inflatable to the same or different volumes as one another. The plurality of dilators may collectively form the shape of any one of the embodiments of the primary balloon described above, for example a trapezoidal prism, a rectangular frustum, and/or a square frustum, when inflated. In embodiments, the plurality of dilators may be configured such that the primary balloon, when inflated, may comprise an aperture extending therethrough. The aperture may be as described previously.

In embodiments, the catheter assembly may comprise an ancillary dilator. The ancillary dilator may be insertable into the same nasal passageway as the primary balloon. The primary balloon may be inflatable to a larger volume than the ancillary dilator. The primary balloon and the ancillary dilator may be connected together. The ancillary dilator may be positionable, in use, above the primary balloon e.g. the ancillary dilator may be positioned adjacent to the roof of the primary balloon and/or between the primary balloon and the roof of the nasal passageway.

Advantageously, this may have a stabilising effect on the primary balloon e.g. aiding alignment during inflation and/or reducing undesirable movement of the primary balloon during use.

In embodiments, the primary balloon and the ancillary dilator may be discrete components. Advantageously, this increases the tunability of the catheter assembly as a surgeon may independently position the primary balloon and the ancillary dilator during a procedure. Advantageously, the ancillary dilator design allows the catheter assembly to be configured to better match the anatomy of the patient's nose. This can be utilized to increase and/or guide the contact between the surfaces of the primary balloon and the ancillary dilator with the nasal cavity and/or the anatomical structures therein e.g., to aid alignment of the primary balloon and ancillary dilator during inflation. This helps prevent the primary balloon from entering the upper part of the nasal cavity and/or moving upwards towards the skull face and the brain.

In embodiments, the primary balloon and ancillary dilator are independently inflatable. Advantageously, this may improve the versatility of the assembly as the surgeon can choose which of and/or to what extent each of the primary balloon and the ancillary dilator inflate and deflate during a procedure.

In embodiments, the primary balloon and ancillary dilator may be simultaneously inflatable. They may reach full inflation at the same time. In some embodiments of the invention, the assembly may be configured such that one of the primary balloon and the ancillary dilator inflates prior to the other. For example, the primary balloon may reach full inflation prior to the ancillary dilator or vice versa. This may be advantageous since the ancillary dilator can be used to apply an additional force to a specific location once the primary balloon has secured the position of the assembly. Alternatively, the ancillary dilator may inflate first e.g. to secure the assembly in position, thereby allowing the primary balloon to apply a force as desired by the user. In embodiments, the catheter assembly may comprise a channel operably connected to the primary balloon. The catheter assembly may comprise a ancillary channel operably connected to the ancillary dilator In alternative embodiments, the catheter assembly may comprise a single channel operably connected to both the primary balloon and the ancillary dilator.. The channel, and optionally, the ancillary channel, may be provided on or in the shaft. The volume (i.e. size) and pressure (i.e. rigidity) of the primary balloon and the ancillary dilator may be adjustable e.g. by selectively inflating the primary balloon and/or the ancillary dilator This means that the amount of pressure exerted on different anatomical structures within the nasal cavity can be controlled with a higher degree of accuracy.

Advantageously, higher pressures can be exerted on the desired anatomical structures such as the septum and/or inferior turbinate to provide a shaping effect, whilst lower pressures can simultaneously be exerted on surrounding anatomical structures and the surrounding mucosa within the nasal cavity to provide a stabilising/aligning effect without damaging these surrounding structures and tissues.

In embodiments, the assembly may comprise two or more ancillary dilators. For example: 2, 3, 4, 5, 6, 7, 8, 9, 10 or more ancillary dilators. Embodiments comprising multiple ancillary dilators can be highly configurable and adaptable in use.

In embodiments, each of the two or more ancillary dilators may be independently inflatable. In embodiments, some or all of the two or more ancillary dilators may be simultaneously inflatable. In embodiments, the catheter assembly may comprise a plurality of ancillary channels. Each channel may be operably connected to each of the ancillary dilators. Alternatively, the catheter assembly may comprise a single ancillary channel operably connected to each of the ancillary dilators. Advantageously, the inclusion of a plurality of independently inflatable ancillary dilators further improves the tunability and versatility of the assembly.

In embodiments, the primary balloon is inflatable in a patient's nasal passageway between the septum and the inferior turbinate to a sufficient volume and pressure to cause the septum and/or inferior turbinate to adopt a desired position and/or configuration.

A desired configuration of the septum may be substantially straight, rather than deviated medially in either direction. A desired position of the septum may be centrally located, such that the two nasal passageways are substantially symmetrical.

A desired position and configuration of the septum may be one where the septum does not protrude inwardly into the nasal passageway to such an extent as to cause blockage of the airways, difficulty breathing, and/or other detrimental effects. A desired position and configuration of the septum may be one where the septum protrudes inwardly into the nasal passageway to a lesser extent than it did previously, so as to at least partially mitigate one or more of the aforementioned effects. A desired position and configuration of the septum may be one that is more straight and/or more centrally located between the two nasal passageways than it previously was.

A desired configuration of the inferior turbinate may be large enough to control the temperature and moisture levels in the nasal passageway but small enough to not cause a blockage of the airways, difficulty breathing, and/or other detrimental effects. A desired configuration of the inferior turbinate may be smaller than it previously was so as to at least partially mitigate one or more of the aforementioned effects.

The sufficient volume and pressure of the primary balloon may be a volume and pressure high enough to cause the balloon to be both rigid and large enough to impart enough force on the septum and/or inferior turbinate, to cause the septum and/or inferior turbinate to adopt a desired position and/or configuration as described above.

In embodiments, the catheter assembly may comprise a secondary shaft and a secondary balloon located at the end thereof. The secondary balloon may be insertable into the opposite nasal passageway to the primary balloon. The secondary balloon may be insertable into the opposite nasal passageway to the nasal passageway being treated.

Advantageously, during an alternative septoplasty procedure, the secondary balloon may be inflated into the nostril which the deviated septum does not protrude into, providing a counterforce to the force exerted on the deviated septum by the primary balloon). This prevents the deviated septum from over-deforming laterally in the opposite direction, beyond the desired central position.

Advantageously, during an alternative procedure for treating an enlarged inferior turbinate, the secondary balloon may be inflated in the nostril that does not contain the enlarged inferior turbinate. This prevents the septum from becoming deviated due to the force exerted on the septum by the primary balloon.

The secondary balloon may be insertable into the same nasal passageway as the primary balloon. The secondary balloon may be insertable into the nasal passageway being treated. In embodiments, the primary balloon, (and the plurality of dilators in such embodiments) may be formed from an elastic mesh material overlaid with a layer of flexible polymer material. In embodiments, the ancillary dilator(s) may be formed from an elastic mesh material overlaid with a layer of flexible polymer material. In embodiments, the secondary balloon may be formed from an elastic mesh material overlaid with a layer of flexible polymer material. Advantageously, using an elastic mesh material does not prevent the primary and/or secondary balloon, and/or plurality of dilators, and/or ancillary dilator(s) from inflating and deflating, whilst reinforcing said inflated balloon(s) and/or dilators to provide the strength required to impart the necessary force on the septum and/or inferior turbinate to achieve a remodelling effect.

Advantageously, overlaying the elastic mesh material with a layer of flexible polymer, allows the primary and/or secondary balloon, and/or a or the plurality of dilators and/or ancillary dilator(s) to inflate and deflate without breaking, and provides the strength required to impart the necessary force on the septum and/or inferior turbinate to achieve a remodelling effect. Furthermore, this layer is fluid tight, enabling the inflation by transmission of a fluid into the balloon(s) and/or dilator(s) from the shaft.

In embodiments, the flexible polymer material may comprise nylon.

In embodiments the catheter assembly may comprise a flexible medical grade material. In embodiments, the catheter shaft may comprise a flexible medical grade material.

In embodiments, the shaft may be made of a material with sufficient strength to allow the catheter assembly to guide the primary balloon into the desired position within the nasal cavity. The desired position within the nasal passageway may be the position wherein, when inflated, the primary balloon will inflate between the septum and the inferior turbinate.

In embodiments, one or more sensors may be positioned on the primary balloon.

In embodiments the catheter assembly may comprise a guide for guiding the primary balloon into the desired position within the nasal passageway.

In embodiments, the catheter assembly may be reusable. For example, the same primary balloon may be used to treat a first nasal passageway of a patient and subsequently, a second nasal passageway of a patient. Additionally or alternatively, the catheter assembly may be sterilised after use and subsequently used to treat a different patient.

Advantageously, reusing the same catheter assembly reduces overall costs of the procedure(s), as fewer devices can treat a greater number of patients and/or conditions. Alternatively, the catheter assembly may be single use. Advantageously, this may improve the hygiene of the procedures, thus reducing the likelihood of infection, particularly in places where sterilisation of medical devices after use may be challenging.

A second aspect of the invention provides a device for use in a septoplasty and/or an inferior turbinate lateralisation procedure comprising the catheter assembly and an inflator configured to inflate at least the primary balloon.

In embodiments, the inflator may comprise a fluid source in fluid communication with the one or more balloons. The inflator may comprise a manual and/or electric pump, syringe, or source of pre-pressurised fluid such as a pressurised gas canister. In embodiments, the inflator and/or fluid source may be controlled by a controller. In embodiments, the inflation of the primary balloon, and in optional embodiments, the plurality of dilators and/or ancillary dilator(s) and/or secondary balloon, may be automatically controlled by a controller. In embodiments, the device may comprise a pressure gauge for monitoring the pressure within the one or more balloons and/or one or more dilators. The pressure inside the primary balloon, and optionally the plurality of dilators, each of the dilators, and/or an ancillary dilator and/or a secondary balloon may be controlled automatically, based on the output of the pressure gauge.

A third aspect of the invention provides a method of treating a deviated nasal septum and/or an enlarged/hypertrophied inferior turbinate of a patient, comprising the steps of:
inserting a catheter assembly of a device having a primary balloon in a deflated state into a first nasal passageway of the patient;
positioning the primary balloon so that it is interposed at least partially between the septum and the inferior turbinate of the patient; and
inflating the primary balloon with fluid until the septum and/or inferior turbinate of the patient adopts a desired position and/or configuration.

Inflating the primary balloon with fluid may comprise inflating the balloon such that the base of the primary balloon contacts the nasal floor of the patient.

Advantageously, this method allows a deviated septum or an enlarged/hypertrophied inferior turbinate to be treated without the need for an overly invasive and complex surgical procedure, which in turn reduces costs and improves patient recovery times.

Further advantageously, the surfaces of the primary balloon contacting/engaging with the nasal floor prevents the primary balloon from moving upwards in the nasal cavity towards the skull face and brain, greatly improving the safety of the method.

However, this method can also cause a deviated septum to over medialize and thus deviate in to the opposite nasal passageway to the nasal passageway initially being treated.

In embodiments, the method further comprises the additional steps of:
deflating the primary balloon;
removing the catheter assembly from the first nasal passageway of the patient;
inserting the catheter assembly into a second nasal passageway of the patient;
positioning the primary balloon so that it is interposed at least partially between the septum and the inferior turbinate of the patient; and
inflating the primary balloon with fluid until the septum and/or inferior turbinate of the patient adopts a desired position and/or configuration.

Inflating the primary balloon with fluid may comprise inflating the balloon such that the base of the primary balloon contacts the nasal floor of the patient.

Advantageously, this allows a deviated septum in one nasal passageway and an enlarged/hypertrophied inferior turbinate in the other nasal passageway to be simultaneously treated in a single procedure, using a single catheter assembly, without the need for one or more overly invasive and complex surgical procedure(s). This is turn reduces costs and improves patient recovery times. Further advantageously, this allows any over medialization to be treated in a simplistic and minimally invasive manner, resulting in reduced costs and improved patient recovery times.

The catheter assembly may be reused. For example, the same primary balloon may be inserted into, inflated and deflated in a first nasal passageway of a patient and subsequently inserted into, inflated and deflated in a second nasal passageway of a patient. Alternatively, the catheter assembly may be sterilised after use, and subsequently used to treat a different patient.

Advantageously, reusing the same catheter assembly reduces overall costs of the procedure(s), as fewer devices can treat a greater number of patients and/or conditions. Alternatively, the catheter assembly may be used to treat a single patient and/or a single nasal passageway.

### BRIEF DESCRIPTION OF SEVERAL VIEWS OF THE DRAWINGS

Advantageously, this may improve the hygiene of the procedures, thus reducing the likelihood of infection. Embodiments of the invention will now be described by way of example only with reference to the accompanying drawings in which:

FIG. 1 is a diagram of the anatomical structures within a nasal cavity of a patient including a nasal septum in a deviated state and an enlarged/hypertrophied inferior turbinate before treatment with a device of the invention;

FIG. 2A is a drawing of an embodiment of a device for performing a septoplasty procedure according to the invention;

FIG. 2B is a drawing of an embodiment of a device for performing a septoplasty procedure according to the invention;

FIG. 2C is a drawing of an embodiment of a device for performing a septoplasty procedure according to the invention; and

FIG. 3 is a diagram of anatomical structures within a nasal cavity of a patient after treatment with a device of the invention.

### DETAILED DESCRIPTION OF THE PREFERRED EMBODIMENTS

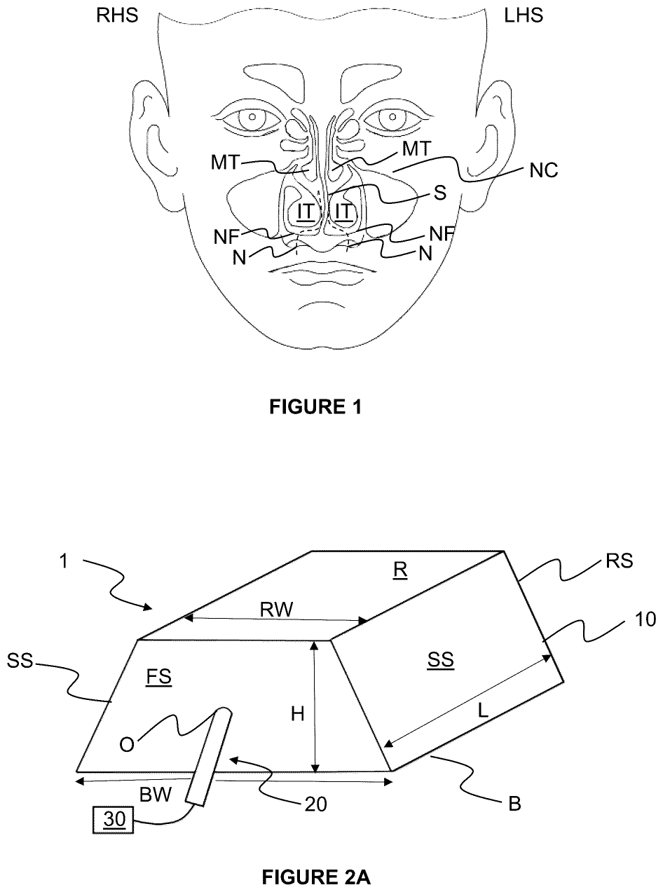

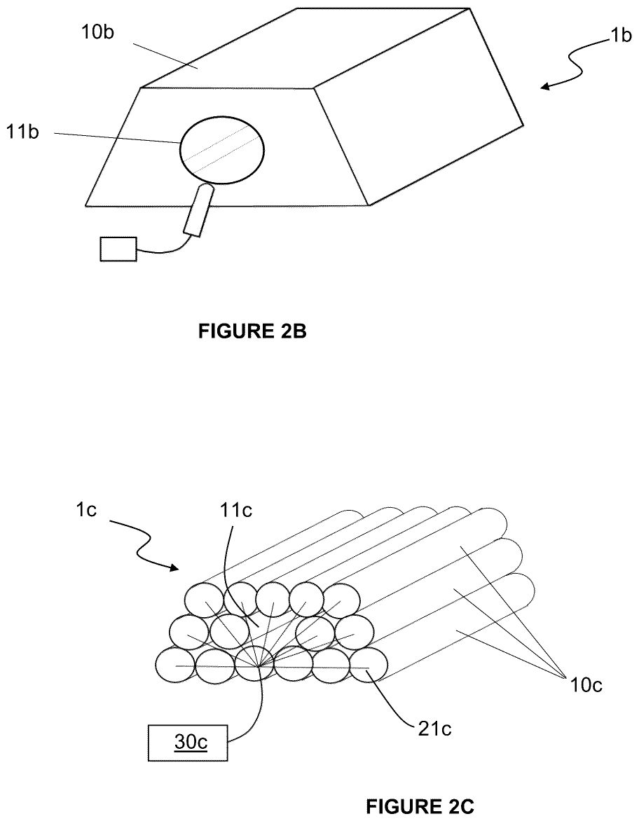

Referring now to FIG. 1, there is shown a diagram of anatomical structures within a nasal cavity NC of a patient prior to treatment with the device of FIG. 2A or 2B. The septum S divides the two air ways of the nasal cavity NC. Here, the septum S is shown in a deviated configuration, deforming laterally towards the patient's left hand side LHS. The inferior turbinates IT and middle turbinates MT are shown on either side of the septum S. The inferior turbinate IT on the right hand side RHS of the patient is enlarged/hypertrophied. On the left hand side of the patient LHS, the deviated septum S causes the septum S and inferior turbinate IT to be in closer proximity to one another, causing a blockage in the air flow path (indicated by the dashed line). On the right hand side of the patient RHS, the enlarged inferior turbinate IT causes the septum S and inferior turbinate IT to be in closer proximity to one another, causing a restriction between these structures, thus reducing the flow rate of the air flow path (indicated by the dashed line).

Referring now to FIG. 2A, there is shown a device 1 for performing a septoplasty procedure and/or a procedure for reducing an enlarged turbinate according to the invention. The device 1 comprises a primary balloon 10 and a shaft 20 operably connected to the primary balloon 10. The shaft 20 is operably connected to a fluid source 30.

When inflated, the primary balloon 10 has the shape of a trapezoidal prism. The inflated primary balloon 10 has a front surface FS and a rear surface RS, each having the shape of a trapezium. The shaft 20 is operably and fluidly connected to the primary balloon 10 via an opening O in the front surface FS. A pair of parallel rectangular surfaces extend between the front surface FS and rear surface RS of the inflated primary balloon 10, defining a roof R and a base B. The base B has a larger surface area than the roof R. In use, the base B may be poisoned atop the nasal floor of the patient in order to reduce slippage of the primary balloon 10 during inflation. A pair of opposing, non-parallel rectangular side surfaces SS extend between the front surface FS and the rear surface RS of the inflated primary balloon 10. In use, these two side surfaces SS respectively contact and exert pressure on the septum and inferior turbinate of the patient, providing a remodelling effect. The relatively flat surfaces provide a significant area over which pressure may be exerted on the nasal tissues to effect the procedure.

The inflated primary balloon 10 has a length L defined as the distance between its front surface FS and rear surface RS of 50 mm. The inflated primary balloon 10 has a height H defined as the distance between the roof and base of 18 mm. The inflated primary balloon 10 has a roof width RW, defined as the width between two edges connecting the roof R to the two side surfaces SS, of 10 mm. The inflated primary balloon 10 has a base width BW, defined as the width between the two edges connecting the base B to the two side surfaces SS, of 14 mm. The base width being greater than the roof width means that, in use, the surfaces of the primary balloon engage with the nasal floor and other nasal structures in such a manner than the balloon is prevented from moving upwards in the nasal cavity towards the skull face and brain. This greatly improves the safety of the catheter assembly and the device.

FIG. 2B shows a further embodiment of catheter assembly 1b wherein description of like components will not be repeated. The primary balloon 10b comprises an aperture 11b which extends the length of the primary ballon 10b and is thus open on the front and rear surfaces of the primary balloon 10b. Such embodiments permit the patient to breathe through the nostril being treated with the catheter assembly 1b.

FIG. 2C shows a further embodiment of catheter assembly 1c wherein description of like components will not be repeated. The primary balloon comprises a plurality of dilators 10c, which in the embodiment shown are approximately cylindrical. Each of the dilators 10c is connected to a fluid source 30c via channels 21c, which are shown schematically. The dilators 10c are connected together such that the primary balloon maintains a trapezium shape when the dilators 10c are inflated. The device is thus able to operate in the same manner as the embodiments described previously. The dilators may be connected together directly and/or secured via one or more supports. An large opening 11c is formed between the dilators 10c to permit the patient to breathe through the nostril being treated in use, although smaller openings may be present between various dilators depending on the shape and ability of the dilators to pack closely together. It would be understood that various shapes and numbers of dilator could be used to achieve the same effect, including multiple different sizes and shapes within a single primary balloon.

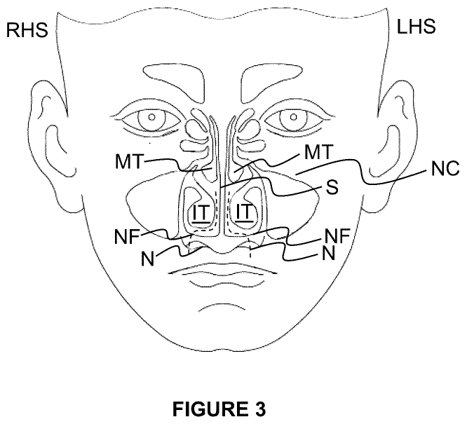

To treat the deviated septum S shown in FIG. 1, the primary balloon 10 of FIG. 2A or 2B in its deflated state is inserted into the nostril N on the patient's left hand side LHS using the shaft 20 and positioned atop the nasal floor NF, and is interposed at least partially between the septum S and the inferior turbinate IT. Once the primary balloon is positioned, fluid is directed from the fluid source 30, through the catheter assembly 20 to the primary balloon 10. Upon inflation, the primary balloon 10 engages the surface of the septum S and the inferior turbinate IT on the left hand side LHS of the patient, causing the septum S to deform medially to the central position shown in FIG. 3.

To treat the enlarged/hypertrophied inferior turbinate IT shown in FIG. 1, the primary balloon 10 of FIG. 2A or 2B in its deflated state is inserted into the nostril N on the patient's right hand side RHS using the catheter assembly 20 and positioned atop the nasal floor NF, and interposed at least partially between the septum S and the inferior turbinate IT. Upon inflation, the primary balloon 10 engages the surface of the septum S and the inferior turbinate IT on the right hand side RHS of the patient, causing the volume of the enlarged/hypertrophied inferior turbinate IT to be reduced by crushing.

Advantageously, the same primary balloon 10 may be used to sequentially treat both the deviated septum S and the enlarged interior turbinate IT. Additionally or alternatively, if the treatment of the deviated septum S causes the septum S to over medialize, and thus deform laterally in the opposite direction of the initial deviation S, the same primary balloon 10 may be inflated in the nostril which the initial deviated septum S protruded into in order to correct the over medialization.

Referring now to FIG. 3, there is shown a diagram of the anatomical structures within a nasal cavity NC of a patient after treatment with the device of FIG. 2A or 2B. The septum S is substantially straight and is located centrally within the nasal cavity NC. The inferior turbinate IT on the patient's right hand side RHS is reduced in volume. As such, the space between the septum S and the inferior turbinate IT on either side of the nasal cavity NC is increased, removing the blockage on the left hand side LHS and increasing the flow rate of the air flow path on the right hand side RHS.

It will be appreciated that the device of the invention affords the medical practitioner with a greater degree of flexibility when treating deviated septa or other nasal medical conditions. The shape of the primary balloon provides a large surface area to exert pressure. The provision of a primary balloon and one or more ancillary dilators allows for a variety of nasal configurations to be accommodated.

While the invention has been described in detail and with reference to specific examples thereof, it will be apparent to one skilled in the art that various changes and modifications can be made therein without departing from the spirit and scope thereof.

What is claimed is:

1. A catheter assembly, insertable into a nasal passageway, for use in a septoplasty, turbinoplasty, and/or septoturbinatoplasty procedure, comprising:
a shaft and a primary balloon;
wherein the primary balloon, when inflated, comprises a base for positioning against the nasal floor, a roof opposed from the base and a side surface extending therebetween for bearing against a nasal septum and/or an inferior turbinate; and
wherein the base has a width greater than the roof in a direction transverse to the axis of the shaft.

2. The catheter assembly according to claim 1, wherein the primary balloon, when inflated, has a shape that tapers from the base to the roof.

3. The catheter assembly according to claim 1, wherein the primary balloon, when inflated, has a lengthwise axis coincident to the axis of the shaft.

4. The catheter assembly according to claim 3, wherein the primary balloon has a trapezium shaped cross-section orthogonal to its lengthwise axis.

5. The catheter assembly according to claim 1, wherein when inflated, the primary balloon comprises a front surface and a rear surface each having a trapezium shape, and a pair of opposing side surfaces comprising non-parallel quadrilateral surfaces.

6. The catheter assembly according to claim 1, wherein when inflated the primary balloon has the shape of a trapezoidal prism or a rectangular frustum.

7. The catheter assembly according to claim 1, wherein when inflated the base has a width of 10-20 mm and/or the roof has a width of 5-15 mm.

8. The catheter assembly according to claim 3, wherein when inflated, the primary balloon has a length in the lengthwise axis of 35-65 mm.

9. The catheter assembly according to claim 1, wherein when inflated the primary balloon has a height defined as the distance between the roof and the base, and wherein the height is 15-35 mm.

10. The catheter assembly according to claim 1, further comprising an ancillary dilator, wherein in use, the ancillary dilator is insertable into the same nasal passageway as the primary balloon.

11. The catheter assembly according to claim 10, wherein the primary balloon is inflatable to a larger volume than the ancillary dilator.

12. The catheter assembly according to claim 10, wherein the primary balloon and ancillary dilator are connected together.

13. The catheter assembly according to claim 10, wherein the primary balloon and the ancillary dilator are independently inflatable.

14. The catheter assembly according to claim 10, wherein the catheter assembly comprises a primary channel operably connected to the primary balloon and a secondary channel operably connected to the ancillary dilator.

15. The catheter assembly according to claim 10, comprising two or more ancillary dilators.

16. The catheter assembly according to claim 1, wherein in use, the primary balloon is positionable in a patient's nasal passageway between the septum and the inferior turbinate and is inflatable to a sufficient volume and pressure to cause the septum and/or inferior turbinate to adopt a desired position and/or configuration.

17. The catheter assembly according to claim 1, wherein the primary balloon is formed from an elastic mesh material overlaid with a layer of flexible polymer material and optionally, wherein the flexible polymer material comprises nylon.

18. A device for use in a septoplasty procedure, comprising the catheter assembly according to claim 1 and an inflator configured to inflate the primary balloon.

19. A method of treating a deviated nasal septum and/or an enlarged/hypertrophied inferior turbinate of a patient, comprising the steps of:
inserting a catheter assembly of claim 1 in a deflated state into a first nasal passageway of the patient;
positioning the primary balloon so that it is interposed at least partially between the septum and the inferior turbinate of the patient; and
inflating the primary balloon with fluid until the septum and/or inferior turbinate of the patient adopts a desired position and/or configuration.

20. A method according to claim 19, comprising the additional steps of:
deflating the primary balloon;
removing the catheter assembly from the first nasal passageway of the patient;
inserting the catheter assembly into a second nasal passageway of the patient;
positioning the primary balloon so that it is interposed at least partially between the septum and the inferior turbinate of the patient;
inflating the primary balloon with fluid until the septum and/or inferior turbinate of the patient adopts a newly desired position and/or configuration.
